(19) United States
(12) Patent Application Publication
(10) Pub. No.: US 2007/0099890 A1
(43) Pub. Date: May 3, 2007

(54) Novel cephalosporin derivatives, its pharmaceutically acceptable salts and manufacturing process thereof

(76) Inventors: Sung-gyu Kim, Daejeon (KR); Jeong-min Lee, Gyeonggi-do (KR); Jong-ok Jeun, Gyeonggi-do (KR); Sang-kwon Sohn, Gyeonggi-do (KR); Kyung-il Chot, Seoul (KR); Joong-hyup Kim, Gyeonggi-do (KR); Ghil-soo Nam, Seoul (KR)

Correspondence Address: NATH & ASSOCIATES, 112 South West Street, Alexandria, VA, 22314, US

(21) Appl. No.: 10/562,125

(22) PCT Filed: Jun. 25, 2004
(86) PCT No.: PCT/KR04/01557

§ 371 Date: Apr. 26, 2006

(30) Foreign Application Priority Data
Jun. 27, 2003 (KR) 10-2003-0042826

(51) Int. Cl. A61K 31/545; C07D 501/14

(52) U.S. Cl. 514/202; 540/222

(57) ABSTRACT

Disclosed herein are novel cephalosporin compounds, pharmaceutically acceptable salts thereof, and a method for preparing the compounds. The compounds show superior antibacterial activity against a wide variety of gram-positive bacteria, including methicillin-resistant Staphylococcus aureus (MRSA) strain. Accordingly, the compounds can be effectively used as antibiotics.

### TECHNICAL FIELD

The present invention relates to novel cephalosporin compounds, pharmaceutically acceptable salts thereof and a method for preparing the compounds. More particularly, the present invention relates to cephalosporin compounds and pharmaceutically acceptable salts thereof, represented by Formula I below:

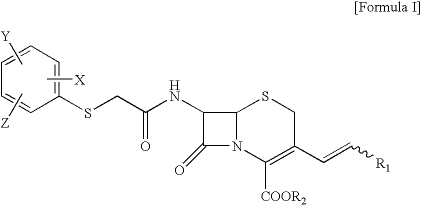

wherein X, Y and Z may be the same or different from one another, and are each independently hydrogen, halogen, C₁₋₆ alkyl, C₁₋₆ alkoxy, C₁₋₆ halogenoalkyl, C₁₋₆ alkoxyalkyl, or C₃₋₆ cycloalkyl;

R₁ is a 3-substituted isoxazolyl group represented by Formula A below:

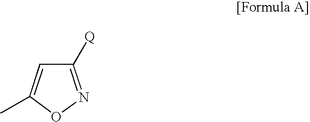

(wherein Q is a substituent useful for the cephalosporin compounds, and is hydrogen, halogen, hydroxy, mercapto, cyano, carboxy, carboxylic acid, ester, carbamoyloxy, carbamoyl, N,N-dimethylcarbamoyl C₁₋₄ alkyl, C₁₋₄ alkyloxy, halogen-substituted alkyl, aryl, or heterocyclic group); and

R₂ is hydrogen, a group forming an ester as a carboxyl derivative, a salt-forming element, or a carboxy-protecting group,
and a method for preparing the compounds.

Since the compounds of Formula I show superior antibacterial activity against a wide variety of gram-positive bacteria, including methicillin-resistant Staphylococcus aureus (MRSA) strain, they can be effectively used as antibiotics.

### BACKGROUND ART

Cephalosporin antibiotics have been widely used in the treatment of infectious diseases caused by pathogenic bacteria in humans and other animals, particularly diseases caused by bacteria resistant to general antibiotics, such as penicillin, and penicillin-hypersensitive patients.

It is widely known that the antibacterial activity of cephalosporin antibiotics is greatly dependent on the kind of substituents present at the 3- and 7-positions of the cephem ring. On the other hand, it is preferable to use antibiotics showing activity against both gram-positive bacteria and gram-negative bacteria in the treatment of infectious diseases. Thus, in order to develop a variety of types antibiotics showing good antibacterial activity against both gram-positive bacteria and gram-negative bacteria, a number of studies on numerous cephalosporin antibiotics in which various substituents are introduced to the 3- or 7-position have been actively undertaken.

In particular, so-called “third-generation cephalosporin antibiotics” such as cefotaxime (U.S. Pat. No. 4,098,888) and cefmenoxime (Japanese Patent Laid-open No. 7675066), and so-called “fourth-generation cephalosporin antibiotics” such as cefepime (U.S. Pat. No. 4,910,301), have been widely used as representative cephalosporin antibiotics. These cephalosporin antibiotics contain an aminothiazolyl-(unsubstituted or substituted) hydroxyiminoacetyl group at the 7-position of the cephem ring, and have a broad antibacterial spectrum against both gram-negative and gram-positive bacteria.

Since an increasing number of gram-positive bacterial species are becoming resistant to drugs and thus are clinically significant, more efficacious therapeutic agents against gram-positive bacteria are urgently needed. It has now been found that the third and fourth generation antibiotics also exhibit unsatisfactory efficacy against resistant strains, potentially causing clinical problems. Thus, there exists a strong need for antibiotics showing strong activity against the resistant strains. In particular, the development of antibiotics showing strong activity against methicillin-resistant Staphylococcus aureus (MRSA) strain has been the focus of intense interest in the art.

In this connection, various cephalosporin antibiotics showing antibacterial activity against methicillin-resistant Staphylococcus aureus (MRSA) strain are already described in many patent publications.

For example, European Patent Laid-open No. 96-72742 discloses cephalosporin compounds showing strong activity against methicillin-resistant Staphylococcus aureus (MRSA) strain, represented by Formula 2 below:

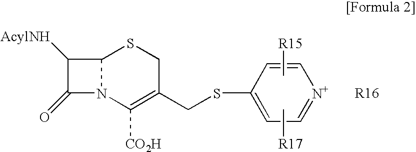

wherein the acyl substituent represents Ar—S—CH₂—CO— (in which Ar is a hydrophobic substituted phenyl, pyridyl, or benzthiazole);

R15 and R16 are each independently hydrogen, alkyl or aminoalkylcarbonylamino; and

R17 is a substituted aliphatic, aromatic, aryl aliphatic, or a group having a sugar moiety.

Specifically, the cephalosporin compounds contain an acyl group at the 7-position of the cephem ring, and a pyridine-based substituent at the 3-position of the cephem ring.

Since the compounds of Formula 2 contain the arylthioacetylamino group (Ar—S—CH₂—CO—) substituted at the 7-position of the cephem ring, they are analogous to the compounds of the present invention in terms of the kind of 7-substituents. On the other hand, the heteroaromatic ring-bonded thioaryl group is substituted at the 3-position of the cephem ring in the compounds of Formula 2, whereas a (3-substituted isoxazol-5-yl)vinyl group is introduced to the 3-position in the compounds of the present invention. Accordingly, the compounds of Formula 2 are different from the compounds of the present invention in terms of the kind of 3-substituents.

To develop cephalosporin compounds showing strong activity against methicillin-resistant Staphylococcus aureus (MRSA) strain, there have been several attempts to introduce an acyl group to the 7-position of the cephem ring and a quaternary ammonium group bonded directly to a propenyl chain to the 3-position of the cephem ring. As a representative example, PCT Publication WO 99-67255 discloses compounds represented by Formula 3 below:

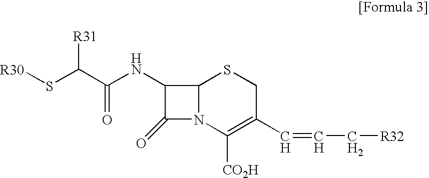

wherein R30 is an organic group having a molecular weight not exceeding 400;

R31 is hydrogen, a lower alkyl or phenyl group; and

R32 is a secondary, tertiary or quaternary amine group bonded directly to the propenyl group and having a molecular weight not exceeding 400.

The compounds of Formula 3 contain various amine groups bonded directly to the propenyl chain at the 3-position of the cephem ring, whereas a (3-substituted isoxazol-5-yl)vinyl group is introduced to the 3-position in the compounds of the present invention. Accordingly, the compounds of Formula 3 are distinguished from the compounds of the present invention in terms of the kind of 3-substituents.

Korean Patent Laid-open No. 2002-5423 describes cephalosporin compounds showing strong antibacterial activity against gram-positive bacteria, e.g., methicillin-resistant Staphylococcus aureus (MRSA) strain, represented by Formula 4 below:

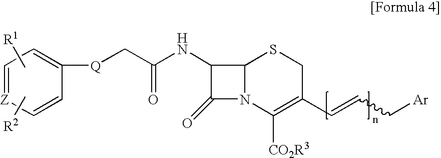

wherein n is an integer of 0 or 1; and

Ar is a heteroary group selected from the following structures:

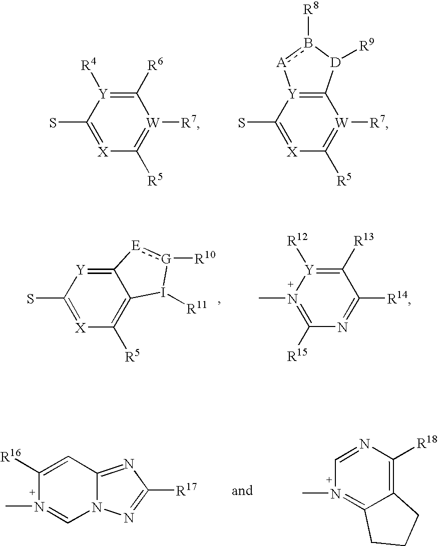

(wherein X, Y, W, A, B, D, E, G and I are each independently N or C, with the proviso that the six-membered ring forms a pyrimidine structure).

The compounds of Formula 4 contain various heteroaromatic rings bonded to the methylene or propenyl group at the 3-position of the cephem ring, whereas an isoxazolyl group is introduced to the 3-position in the compounds of the present invention. Accordingly, the compounds of Formula 4 are distinguished from the compounds of the present invention in terms of the kind of 3-substituents.

In conclusion, none of the above patent publications disclose cephalosporin compounds containing a (3-substituted isoxazol-5-yl)vinyl group at the 3-position of the cephem ring, like the compounds of the present invention.

### DISCLOSURE

### Technical Problem

Thus, the present inventors have earnestly and intensively conducted research to develop cephalosporin compounds having a broad antibacterial activity against gram-positive bacteria, including methicillin-resistant Staphylococcus aureus (MRSA) strain, and as a result, have found that cephalosporin compounds containing a (3-substituted isoxazol-5-yl)vinyl group at the 3-position of the cephem ring have a broad antibacterial activity against gram-positive bacteria. The present invention is based on this finding.

Therefore, it is an object of the present invention to provide novel cephalosporin compounds and pharmaceutically acceptable salts thereof, represented by Formula I.

It is another object of the present invention to provide a method for preparing the cephalosporin compounds or their salts.

It is yet another object of the present invention to provide an antibacterial composition comprising the compound of Formula I as an active ingredient.

### TECHNICAL SOLUTION

In accordance with one aspect of the present invention, the above objects can be accomplished by cephalosporin compounds and pharmaceutically acceptable salts thereof, represented by Formula I below:

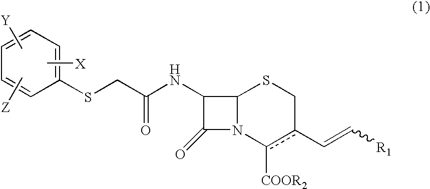

wherein X, Y and Z may be the same or different from one another, and are each independently hydrogen, halogen, C₁₋₆ alkyl, C₁₋₆ alkoxy, C₁₋₆ halogenoalkyl, C₁₋₆ alkoxyalkyl, or C₃₋₆ cycloalkyl;

R₁ is a 3-substituted isoxazolyl group represented by Formula A below:

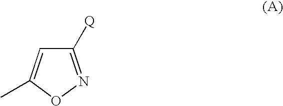

(wherein Q is a substituent useful for the cephalosporin compounds, and is hydrogen, halogen, hydroxy, mercapto, cyano, carboxy, carboxylic acid, ester, carbamoyloxy, carbamoyl, N,N-dimethylcarbamoyl, C₁₋₄ alkyl, C₁₋₄ alkyloxy, halogen-substituted alkyl, aryl, or heterocyclic group); and

R₂ is hydrogen, a group forming an ester as a carboxyl derivative, a salt-forming element, or a carboxy-protecting group.

As described above, the compounds of Formula I according to the present invention are cephalosporin compounds, characterized by the introduction of a (3-substituted isoxazol-5-yl)vinyl group at the 3-position of the cephem ring. As apparent from the following Examples and test Examples, the compounds of Formula I show superior antibacterial activity against gram-positive bacteria, including methicillin-resistant Staphylococcus aureus (MRSA) strain.

Detailed description will be made of the compounds of Formula I according to the present invention.

As described previously, the substituent R₁ of the compounds of Formula I is a 3-substituted isoxazolyl group represented by the above Formula A wherein Q is a substituent useful for the cephalosporin compounds, and is hydrogen, halogen, C₁₋₄ alkyl, C₁₋₄ alkyloxy, halogen-substituted alkyl aryl, heterocyclic substituent or the like.

The term “halogen” used herein refers to fluorine, chlorine, bromine, or iodine.

The heterocylic substituent is an unsaturated 5- or 6-membered hetero ring which contains at least one atom selected from oxygen, sulfur and nitrogen. Representative examples of heterocyclic substituents include unsubstituted or substituted thiazolylthio, isothiazolylthio, thiadiazolylthio, triazolylthio, triazinylthio, tetrazolylthio, triazolopyrimidinylthio, 1-substituted pyridiniumthio-4-yl-thio, etc. The pyrimidinium group may be substituted with C₁₋₆ alkyl, hydroxyalkyl, alkoxyalkyl, carboxyalkyl, sulfonylalkyl, carbamoylalkyl, and aminoalkyl groups at its 1-position. The aminoalkyl group may be unsubstituted or substituted with one or two substituents.

The substituent R₂ in the compounds of Formula I according to the present invention may be hydrogen, a group forming an ester as a carboxyl derivative, a salt-forming element, or a carboxy-protecting group. The carboxy-protecting group is a functional group which can be readily introduced to or removed from the cephalosporin compounds, without adversely affecting other positions of the molecules. Illustrative of suitable carboxy-protecting groups include unsubstituted or substituted C₁₋₈ alkyl groups (e.g., methyl, methoxymethyl, ethyl, methoxyethyl, propyl, isopropyl, butyl, isobutyl, t-butyl and hexyl ester), and aryl groups (e.g., phenyl, indanyl, benzyl, cyanobenzyl, halobenzyl, methylbenzyl, nitrobenzyl, para-methoxybenzyl and phenylbenzyl), and the like.

Further, in the case where the substituent R₂ in the compounds of the present invention is a group forming an ester as a carboxyl derivative, the compounds of the present invention have an ester structure (i.e. carboxyl derivatives), which can show in vivo antibiotic activity when administered in the form of an oral or injectable preparation. As the carboxylic derivatives, there may be mentioned, for example, well-known 1-substituted C₃₋₁₂ alkyl esters, alkanoyloxyalkyl esters (more specifically, acetoxymethyl, acetoxyethyl, propionyloxyethyl, pivaloyloxyethyl, tetrahydrofuryl, and tetrahydropyranyl esters), C₃₋₈ alkoxyformyloxyalkyl esters (e.g., ethoxycarbonyloxy esters), substituted C₇₋₁₅ aralkyl esters (e.g., penasil, indanyl esters), 2-alkenylesters (e.g., allyl, 2-oxo-1,3-dioxol-4-ylmethyl esters), and the like.

As the salt-forming element, any element that can form inorganic or organic salts of the cephalosporin compounds may be used. Representative inorganic salts are sodium and potassium salts, and organic salts are salts of alkylamines (e.g., lower alkylamines such as ethylamine, diethylamine, and triethylamine), salts of aromatic amines (e.g., aniline, and diethylaniline), and salts of aromatic bases (e.g., picoline, lutidine, and quinoline), and the like.

Depending on the structure of the vinyl group at the 3-position of the cephem ring, the compounds of Formula I according to the present invention can be present in the cis- or trans-form, which are within the scope of the present invention, represented by Formulae Ia and Ib:

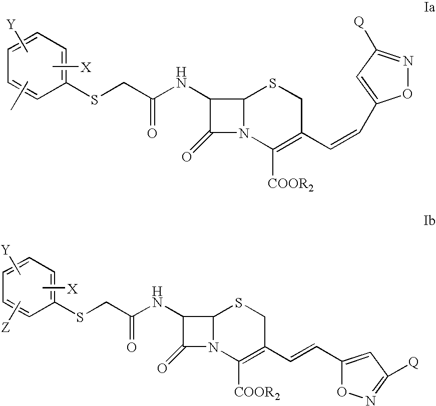

wherein X, Y, Z, Q and R₂ are as defined above.

Representative examples of the compounds according to the present invention include:
Compound 1: para-methoxybenzyl(6R,7R)-7-phenylthioacetamido-3-[(3-methylisoxazol-5-yl)vinyl]-3-cephem-4-carboxylate;
Compound 2: para-methoxybenzyl(6R,7R)-7-phenylthioacetamido-3-[(3-ethylisoxazol-5-yl)vinyl]-3-cephem-4-carboxylate;
Compound 3: para-methoxybenzyl(6R,7R)-7-phenylthioacetamido-3-[(3-methoxyisoxazol-5-yl)vinyl]-3-cephem-4-carboxylate;
Compound 4: para-methoxybenzyl(6R,7R)-7-phenylthioacetamido-3-[(3-ethoxyisoxazol-5-yl)vinyl]-3-cephem-4-carboxylate;
Compound 5: para-methoxybenzyl(6R,7R)-7-phenylthioacetamido-3-[(3-bromoisoxazol-5-yl)vinyl]-3-cephem-4-carboxylate;
Compound 6: para-methoxybenzyl(6R,7R)-7-phenylthioacetamido-3-[(3-hydroxyisoxazol-5-yl)vinyl]-3-cephem-4-carboxylate;
Compound 7; para-methoxybenzyl(6R,7R-7-phenylthioacetamido-3-[(3-ethoxycarbonylisoxazol-5-yl)vinyl)-3-cephem-4-carboxylate;
Compound 8: para-methoxybenzyl(6R,7R)-7-phenylthioacetamido-3-[(3-phenylisoxazol-5-yl)vinyl]-3-cephem-4-carboxylate;
Compound 9: para-methoxybenzyl(6R,7R)-7-phenylthioacetamido-3-[[3-(4-methylphenyl)isoxazol-5-yl]vinyl]-3-cephem-4-carboxylate;
Compound 10: para-methoxybenzyl(6R,7R)-7-phenylthioacetamido-3-[[3-(4-methoxyphenyl)isoxazol-5-yl]vinyl]-3-cephem-4-carboxylate;
Compound 11: para-methoxybenzyl(6R,7R-7-phenylthioacetamido-3-[(3-(4-fluorophenyl)isoxazol-5-yl]vinyl]-3-cephem-4-carboxylate;
Compound 12: para-methoxybenzyl(6R,7R)-7-phenylthioacetamido-3-[[3-(4-chlorophenyl)isoxazol-5-yl]vinyl]-3-cephem-4-carboxylate,
Compound 13: para-methoxybenzyl(6R,7R)-7-phenylthioacetamido-3-([[3-(4-bromophenyl)isoxazol-5-yl]vinyl]-3-cephem-4-carboxylate;
Compound 14: para-methoxybenzyl(6R,7R)-7-phenylthioacetamido-3-[[3-(pyridin-2-yl)isoxazol-5-yl]vinyl]-3-cephem-4-carboxylate;
Compound 15: para-methoxybenzyl(6R,7R)-7-phenylthioacetamido-3-[[3-(pyridin-3-yl)isoxazol-5-yl]vinyl]-3-cephem-4-carboxylate;
Compound 16: para-methoxybenzyl(6R,7R)-7-phenylthioacetamido-3-[[3-(pyridin-4-yl)isoxazol-5-yl]vinyl]-3-cephem-4-carboxylate;
Compound 17: para-methoxybenzyl(6R,7R)-7-phenylthioacetamido-3-[(3-carbamoylisoxazol-5-yl)vinyl]-3-cephem-4-carboxylate;
Compound 18: (6R,7R)-7-phenylthioacetamido-3-[(3-methylisoxazol-5-yl)vinyl]-3-cephem-4-carboxylic acid;
Compound 19: (6R,7R)-7-phenylthioacetamido-3-[(3-ethylisoxazol-5-yl)vinyl]-3-cephem-4-carboxylic acid;
Compound 20: (6R,7R)-7-phenylthioacetamido-3-[(3-methoxyisoxazol-5-yl)vinyl]-3-cephem-4-carboxylic acid;
Compound 21: (6R,7R-7-phenylthioacetamido-3-[(3-ethoxyisoxazol-5-yl)vinyl]-3-cephem-4-carboxylic acid;
Compound 22: (6R,7R)-7-phenylthioacetamido-3-[(3-bromoisoxazol-5-yl)vinyl]-3-cephem-4-carboxylic acid;
Compound 23: (6R,7R)-7-phenylthioacetamido-3-[(3-hydroxyisoxazol-5-yl)vinyl]-3-cephem-4-carboxylic acid;
Compound 24: (6R,7R)-7-phenylthioacetamido-3-[(3 ethoxycarbonylisoxazol-5-yl)vinyl]-3-cephem-4-carboxylic acid;
Compound 25: (6R,7R)-7-phenylthioacetamido-3-[(3-phenylisoxazol-5-yl)vinyl]-3-cephem-4-carboxylic acid;
Compound 26: (6R,7R)-7-phenylthioacetamido-3-[[3-(4-methylphenyl)isoxazol-5-yl]vinyl]-3-cephem-4-carboxylic acid;
Compound 27: (6R,7R)-7-phenylthioacetamido-3-[[3-(4-methoxyphenyl)isoxazol-5-yl]vinyl]-3-cephem-4-carboxylic acid;
Compound 28: (6R,7R)-7-phenylthioacetamido-3-[[3-(4-fluorophenyl)isoxazol-5-yl]vinyl]-3-cephem-4-carboxylic acid;
Compound 29: (6R,7R-7-phenylthioacetamido-3-[[3-(4-chlorophenyl)isoxazol-5-yl]vinyl]-3-cephem-4-carboxylic acid;
Compound 30: (6R,7R-7-phenylthioacetamido-3-[[3-(4-bromophenyl)isoxazol-5-yl]vinyl]-3-cephem-4-carboxylic acid;
Compound 31: (6R,7R)-7-phenylthioacetamido-3-[[3-(pyridin-2-yl)isoxazol-5-yl]vinyl]-3-cephem-4-carboxylic acid;
Compound 32: (6R,7R)-7-phenylthioacetamido-3-[[3-(pyridin-3-yl)isoxazol-5-yl]vinyl]-3-cephem-4-carboxylic acid;
Compound 33: (6R,7R)-7-phenylthioacetamido-3-[[3-(pyridin-4-yl)isoxazol-5-yl]vinyl]-3-cephem-4-carboxylic acid; and
Compound 34: (6R,7R)-7-phenylthioacetamido-3-[(3 carbamoylisoxazol-5-yl)vinyl]-3-cephem-4-carboxylic acid.

In accordance with another aspect of the present invention, there is provided a method for manufacturing the cephalosporin compounds or pharmaceutically acceptable salts thereof, comprising the steps of preparing the compound represented by Formula I below:

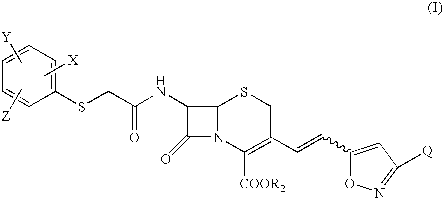

wherein X, Y, Z, R₂ and Q are as defined above,

by reacting an ylide of Formula VI below:

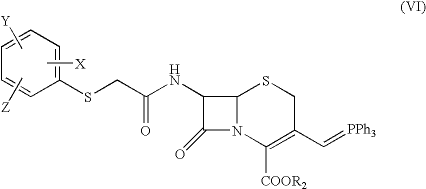

wherein X Y, Z and R₂ are as defined above,

with an aldehyde compound of Formula VII below:

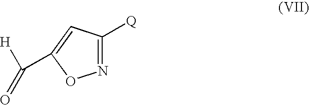

wherein Q is as defined above,

in the presence of a base and an organic solvent.

Hereinafter, the method of the compounds according to the present invention will be explained in more detail. The compounds of the present invention can be prepared by introducing a (3-substituted isoxazol-5-yl)vinyl group to the 3-position of the cephem ring in the compound of Formula VI, via the Wittig reaction. In the method of the present invention, any of conventional bases that have been used for the Wittig reaction may be used. It is preferable to use at least one base selected from the group consisting of sodium carbonate, sodium hydrogen carbonate, alkali metal hydride, alkali metal amide, alkali metal hydroxide, alkali metal acetate, tri-(lower)alkylbenzylamine, N-lower alkylmorpholine, N,N-(lower)alkylbenzylamine and N,N-di-(lower)alkylaniline.

Further, the solvent used in the method of the present invention is preferably at least one selected from the group consisting of water, acetone, dioxane, acetonitrile, chloroform, dichloromethane, tetrahydrofuran, ethylacetate and N,N-dimethylformamide. Further, the reaction temperature is preferably between −40° C. and 25° C.

The compound of Formula VI, which is used as a starting material in the method of the present invention, can be prepared in accordance with the following procedure. First, a compound of Formula IV is reacted with a mixture of a trialkylphosphine (e.g., triphenylphosphine or tributylphosphine) and a sodium or potassium halide (e.g., sodium iodide, sodium chloride, potassium iodide or potassium chloride) in the presence of a solvent, to prepare a phosphonium compound of Formula V, as depicted in Reaction Scheme 1 below:

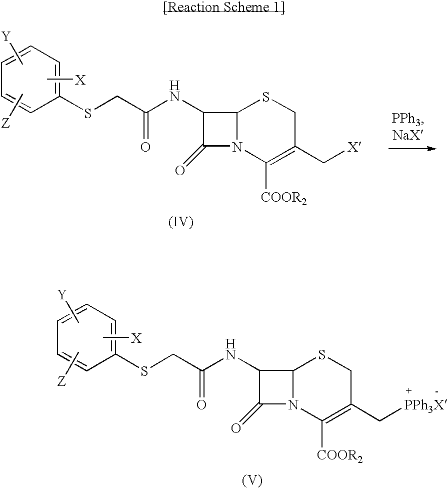

wherein X, Y, Z and R₂ are as defined above, and X′ is chloro, bromo or iodo.

Then, the compound of Formula V is treated with a base to prepare the compound of Formula VI, as depicted in Reaction Scheme 2 below:

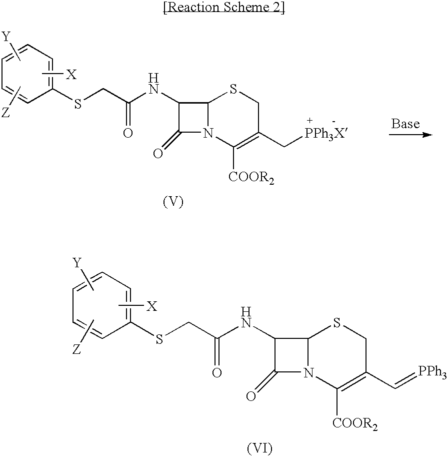

wherein X, Y, Z, R₂ and X′ are as defined above.

In the preparation of the compound of Formula V as depicted in Reaction Scheme 1, acetone, dioxane, acetonitrile, chloroform dichloromethane, tetrahydrofuran, ethylacetate, N,N-dimethylformamide, or the like is preferably used as the solvent. Of these, acetone is particularly preferred. Furthers the preparation reaction of the compound of Formula V is carried out at a temperature of −20° C. to 25° C., and preferably 10° C. to 25° C.

The compound of Formula IV as a starting material of the compound of Formula V depicted in Reaction Scheme 1 can be prepared by reacting a carboxylic acid compound with a cephalosporin compound of Formula III, as depicted in Reaction Scheme 3 below:

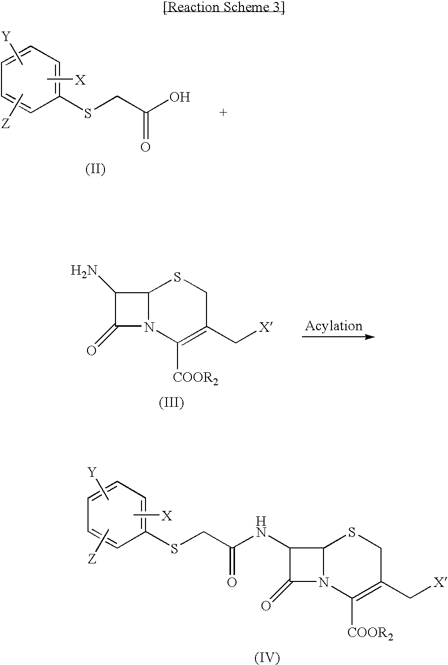

wherein X, Y, Z, R₂ and X′ are as defined above.

In the preparation of the compound of Formula IV, an acylation reaction between the carboxylic acid compound of Formula II and the cephalosporin compound of Formula III is conducted by a routine reaction process. As the reaction solvent, water, acetone, dioxane, acetonitrile, chloroform, dichloromethane, tetrahydrofuran, ethylacetate, N,N-dimethylformamide, or pyridine can be preferably used.

The acylation reaction is conducted in the presence of an organic or inorganic base. Examples of bases usable herein include alkali metal hydroxide, alkali metal acetate, tri-(lower)alkylamine, pyridine, N-lower alkylmorpholine, N,N-(lower)alkylbenzylamine and N,N-di-(lower)alkylaniline. The acylation reaction is preferably conducted at a temperature between −40° C. and 25° C.

In addition, the compound of Formula II is required to be activated using an activating agent for the acylation reaction. Examples of activating agents include acyl halides, acyl azides, activating esters, activating amides, and acid anhydrides (including symmetric or mixed anhydrides). As compounds forming the acid anhydrides, there may be mentioned, for example, inorganic acids (e.g., phosphoric acid, sulfuric acid, halogen acids, etc.), and organic acids (e.g., alkane acids, aralkane acids, alkylsulfonic acids, arylsulfonic acides, etc.). In addition, the acylation reaction can be conducted using a coupling assistant, e.g., N,N-dicyclohexylcarbodiimide, N-cyclohexyl-N-(4-diethylaminocyclohexyl)carbodiimide, N,N-carbonylbis(2-methylimidazole), ethyl polyphosphate, phosphorous trichloride, thionyl chloride, oxalyl chloride, triphenylphosphine, or the like.

In this manner, the compound of Formula I according to the present invention is prepared. If necessary, the protecting group is removed by reacting the protected compound of the present invention with an acid, affording the deprotected product, as depicted in Reaction Scheme 4 below:

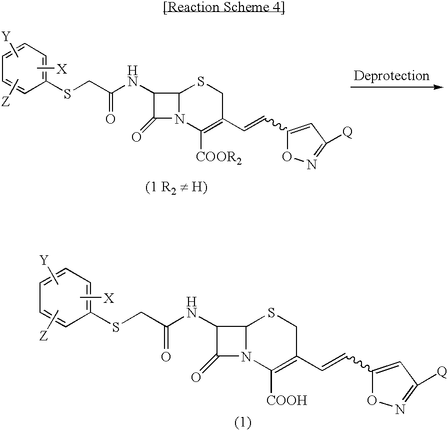

wherein X, Y, Z, R₂ and Q are as defined above.

As the acid used herein, acetic acid, formic acid, trifluoroacetic acid, or a Lewis acid (e.g., aluminum trichloride) is preferred. The amount of the acid to be added is preferably 1˜1,000 times, and more preferably 5˜100 times, that of the cephalosporin compound of Formula I. Further, the deprotection reaction is typically conducted at a temperature between −10° C. and 25° C.

The compounds of Formula I thus prepared can be purified to high purity by various processes, such as recrystallization, iontophoresis, silica gel chromatography, and ion-exchange resin chromatography.

If needed, the substituent R₂ in the compounds of the present invention may be a group forming an ester as a carboxyl derivative. In this case, since the compounds of the present invention have an ester structure (i.e. carboxyl derivatives), they can show in vivo antibiotic activity when administered in the form of an oral or injectable preparation.

This preparation of the carboxyl derivative can be carried out by conventional procedures. Exemplary procedure is specifically described in the following Example 5.

In accordance with yet another aspect of the present invention, there is provided an antibiotic composition comprising the compound or its pharmaceutically acceptable salt of Formula I, and a pharmaceutically acceptable carrier.

Depending on the desired application, the compound of the present invention can be administered in the form of an oral or injectable preparation. The preparation can be obtained using known carriers and excipients by conventional processes known in the art. The preparation may be in the form of a solution, suspension or emulsion in an oil or aqueous medium, and may contain an additive, such as a dispersing, suspending or stabilizing agent. In addition, the preparation may be in a dry powder form, which is dissolved in sterilely treated or pyrogen-free water prior to use. The compounds of Formula I can be formulated into suppositories using common bases such as cocoa butter and glyceride.

Solid dose forms of the compounds according to the present invention for oral administration may be capsules, tablets, pills, powders and granules. The tablets and pills may be covered with enteric coating. In addition, the solid dose forms can be manufactured by mixing the active compound of Formula I with at least one inert diluent selected from sucrose, lactose and starch, and a carrier selected from lubricants (e.g., magnesium stearate), disintegrants and binders.

If necessary, the compounds of the present invention can be combined with penicillin or other cephalosporin antibiotics before being administered.

### MODE FOR INVENTION

The invention will now be more particularly illustrated by reference to the preferred experimental examples. The experimental examples, however, do not limit the scope of the invention, but simply exemplify the preparation of the compounds of the invention.

### REFERENCE Example 1

### Preparation of para-methoxybenzyl(6R,7R)-phenylthioacetamido-3-chloromethyl-3-cephem-4-carboxylate [Process for the Preparation of the Compound (IV)]

para-Methoxybenzyl (6R,7R)-7-amino-3-chloromethyl-3-cephem-4-carboxylate hydrochloride (4.05 g, 10 mmol) was suspended in methylene chloride (80 mL) and the mixture was cooled to 0°. To the suspension was added N,N-diisopropylamine (2.1 mL, 12 mmol) and the resulting mixture was stirred for 10 min. Then, phenylthioacetyl chloride (12 mmol) was added and stirred for 30 min. After completion of the reaction, the reaction mixture was washed with water and brine, dried with anhydrous magnesium sulfate, and then filtered. The filtrate was evaporated under vacuum to give the title compound (5.13 g, 98.6%).

¹H NMR (CDCl₃, 300 MHz, δ): 7.56 (d, 1H), 7.29 (m, 7H), 6.90 (d, 2H), 5.80 (dd, 1H), 5.23 (s, 2H), 4.92 (d, 1H), 4.46 (dd, 2H), 3.81 (s, 3H), 3.49 (dd, 2H)

### Preparation of para-Methoxyphenol(6R,7R)-7-(3,4-dichlorophenyl)thioacetamido-3-chloromethyl-3-cephem-4-carboxylate

The title compound (4.93 g, 85%) was obtained by following the same procedure as the above-mentioned Reference example 1 using 3,4-dichlorophenylthioacetyl chloride (12 mmol) instead of phenylthioacetyl chloride.

¹H NMR (CDCl₃, 300 MHz, δ): 7.45 (s, 1H), 7.29 (m, 5H), 6.90 (d, 2H), 5.80 (dd, 1H), 5.23 (s, 2H), 4.92 (d, 1H), 4.46 (dd, 2H), 3.81 (s, 3H), 3.49 (dd, 2H)

### Preparation of para-Methoxybenzyl(6R,7R)-7-(3,5-dimethylphenyl)thioacetamido-3-chloromethyl-3-cephem-4-carboxylate

The title compound (4.90 g, 87%) was obtained by following the same procedure as the above-mentioned Reference example 1 using 3,5-dimethylphenylthioacetyl chloride (12 mmol) instead of phenylthioacetyl chloride.

¹H NMR (CDCl₃, 300 MHz, δ): 7.45 (s, 1H), 7.29 (m, 5H), 6.90 (d, 2H), 5.80 (dd, 1H), 5.23 (s, 2H), 4.92 (d, 1H), 4.46 (dd, 2H), 3.81 (s, 3H), 3.49 (dd, 2H)

### REFERENCE EXAMPLE 2

### Preparation of para-methoxybenzyl(6R,7R)-7-phenylthioacetamido-3-triphenylphosphoniummethyl-3-cephem-4-carboxylate iodide [Process for the Preparation of the Compound (V)]

Triphenylphosphine (1.57 g, 6 mmol) and sodium iodide (0.90 g, 6 mmol) were added to a solution of para-Methoxybenzyl (6R,7R)-7-phenylthioacetamido-3-chloromethyl-3-cephem-4-carboxylate (2.60 g, 5 mmol) in acetone (100 mL), and the resulting mixture was stirred for 2 h at room temperature. The reaction mixture was filtered, and the filtrate was concentrated and dried under-vacuum to provide the title compound (4.15 g, 95%).

### Preparation of para-Methoxybenzyl(6R,7R)-7-(3,4-dichlorophenyl)thioacetamido-3-triphenylphosphoniummethyl-3-cephem-4-carboxylate iodide

The title compound (4.40 g, 96%) was obtained by following the same procedure as the above-mentioned Reference example 2 using para-methoxybenzyl(6R,7R)-7-(3,4-dichlorophenyl)thioacetamido-3-chloromethyl-3-cephem-4-carboxylate as the reactant.

¹H NMR (CDCl₃, 300 MHz, δ): 7.85 (d, 2H), 7.60 (m, 20H), 6.90 (d, 2H), 5.90 (d, 1H), 5.25 (s, 2M, 4.95 (d, 1H), 4.46 (dd, 2H), 3.84 (s, 3H), 3.50 (dd, 2H)

### Preparation of para-Methoxybenzyl(6R,7R)-7-(3,5-dimethylphenyl)thioacetamido-3-triphenylphosphoniummethyl-3-cephem-4-carboxylate iodide

The title compound (4.18 g, 95%) was obtained by following the same procedure as the above-mentioned Reference example 2 using para-methoxybenzyl(6R,7R)-7-(3,5-dimethylphenyl)thioacetamido-3-chloromethyl-3-cephem-4-carboxylate as the reactant.

¹H NMR (CDCl₃, 300 MHz, δ): 7.85 (d, 2H), 7.60 (m, 20H), 6.90 (d, 2H), 5.90 (d, 1H), 5.25 (s, 2H), 4.95 (d, 1H), 4.46 (dd, 2H), 3.84 (s, 3H), 3.50 (dd, 2H), 2.2 (s, 6H)

### EXAMPLE 1

### Preparation of para-methoxybenzyl(6R,7R)-7-phenylthioacetamido-3-[(3-methylisoxazol-5-yl)vinyl]-3-cephem-4-carboxylate [Process for the Preparation of the Compound (I)]

para-Methoxybenzyl(6R,7R)-7-phenylthioacetamido-3-triphenylphosphoniummethyl-3-cephem-4-carboxylate iodide (437 mg, 0.5 mmol) and 3-methylisoxazol-5-carbaldehyde (56 mg, 0.5 mmol) were suspended in methylene chloride (5 mL), and was added 5% aqueous sodium bicarbonate solution (1 mL). After stirring 5 h at room temperature, the reaction mixture was washed with water and brine, dried with anhydrous magnesium sulfate, filtered, and concentrated under vacuum. The residue was then column chromatographed on silica gel, eluting with a 1:2 mixture of ethyl acetate and n-hexane, to provide the title compound (195 mg, 70.1%).

¹H NMR (CDCl₃, 300 MHz, δ): 7.50 (d, 2H), 7.32 (m, 7H), 6.87 (d, 2H), 6.63 (d, 1H), 6.38 (d, 1H), 6.03 (s, 1H), 5.85 (dd, 1H), 5.16 (d, 2H), 5.04 (d, 1H), 3.81 (s, 3H), 3.71 (q, 2H), 3.42 (dd, 2H), 2.82 (s, 3H)

### Preparation of para-Methoxybenzyl(6R,7R)-7-(3,4-dichlorophenyl)thioacetamido-3-[(3-methylisoxazol-5-yl)vinyl]-3-cephem-4-carboxylate

The title compound (240 mg, 81%) was obtained by following the same procedure as the above-mentioned Example 1 using the corresponding amount of para-methoxybenzyl(6R,7R)-7-(3,4-dichlorophenyl)thioacetamido-3-triphenylphosphoniummethyl-3-cephem-4-carboxylate iodide as the reactant.

¹H NMR (CDCl₃, 300 MHz, δ): 7.50 (d, 2H), 7.32 (m, 5H), 6.87 (d, 2H), 6.63 (d, 1H), 6.38 (d, 1H), 6.03 (s, 1H), 5.85 (dd, 1H), 5.16 (d, 2H), 5.04 (d, 1H), 3.81(s, 3H), 3.71(q, 2H), 3.42(dd, 211, 2.82(s, 3H)

### Preparation of para-Methoxybenzyl(6R,7R)-7-(3,5-dimethylphenyl)thioacetamido-3-[(3-methylisoxazol-5-yl)vinyl]-3-cephem-4-carboxylate

The title compound (220 mg, 80%) was obtained by following the sane procedure as the above-mentioned Example 1 using the corresponding amount of para-methoxybenzyl(6R,7R)-7-(3,5-dimethylphenyl)thioacetamido-3-triphenylphosphoniummethyl-3-cephem-4-carboxylate iodide as the reactant.

¹H NMR (CDCl₃, 300 MHz, δ): 7.50 (d, 2H), 7.32 (m, 5H), 6.87 (d, 2H), 6.63 (d, 1H), 6.38 (d, 1H), 6.03 (s, 1H), 5.85 (dd, 1H), 5.16 (d, 2H), 5.04 (d, 1H), 3.81(s, 3H), 3.71(q, 2H), 3.42(dd, 2H), 2.82(s, 3H), 2.2(s, 6H)

### EXAMPLE 2

The following compounds were prepared by the same procedure as the Example 1 using the corresponding carbaldehydes instead of 3-methylisoxazol-5-carbaldehyde.
1) para-Methoxybenzyl(6R,7R)-7-phenylthioacetamido-3-[(3-ethylisoxazol-5-yl)vinyl]-3-cephem-4-carboxylate
2) para-Methoxybenzyl(6R,7R)-7-phenylthioacetamido-3-[(3-methoxyisoxazol-5-yl)vinyl]-3-cephem-4-carboxylate
3) para-Methoxybenzyl(6R,7R)-7-phenylthioacetamido-3-[(3-ethoxyisoxazol-5-yl)vinyl]-3-cephem-4-carboxylate
4) para-Methoxybenzyl(6R,7R)-7-phenylthioacetamido-3-[(3-bromoisoxazol-5-yl)vinyl]-3-cephem-4-carboxylate
5) para-Methoxybenzyl(6R,7R)-7-phenylthioacetamido-3-[(3-hydroxyisoxazol-5-yl)vinyl]-3-cephem-4-carboxylate
6) para-Methoxybenzyl(6R,7R)-7-phenylthioacetamido-3-[(3-ethoxycarbonylisoxazol-5-yl)vinyl]-3-cephem-4-carboxylate
7) para-Methoxybenzyl(6R,7R)-7-phenylthioacetamido-3-[(3-phenylisoxazol-5-yl)vinyl]-3-cephem-4-carboxylate
8) para-Methoxybenzyl(6R,7R)-7-phenylthioacetamido-3-[[3-(4-methylphenyl)isoxazol-5-yl]vinyl]-3-cephem-4-carboxylate
9) para-Methoxybenzyl(6R,7R)-7-phenylthioacetamido-3-[[3-(4-ethoxyphenyl)isoxazol-5-yl]vinyl]-3-cephem-4-carboxylate
10) para-Methoxybenzyl(6R,7R)-7-phenylthioacetamido-3-[[3-(4-fluorophenyl)isoxazol-5-yl]vinyl]-3-cephem-4-carboxylate
11) para-Methoxybenzyl(6R,7R)-7-phenylthioacetamido-3-[[3-(4-chlorophenyl)isoxazol-5-yl]vinyl]-3-cephem-4-carboxylate
12) para-Methoxybenzyl(6R,7R)-7-phenylthioacetamido-3-[[3-(4-bromophenyl)isoxazol-5-yl]vinyl]-3-cephem-4-carboxylate
13) para-Methoxybenzyl(6R,7R)-7-phenylthioacetamido-3-[[3-(2-pyridinyl)isoxazol-5-yl]vinyl]-3-cephem-4-carboxylate
14) para-Methoxybenzyl(6R,7R)-7-phenylthioacetamido-3-[[3-(3-pyridinyl)isoxazol-5-yl]vinyl]-3-cephem-4-carboxylate
15) para-Methoxybenzyl(6R,7R)-7-phenylthioacetamido-3-[[3-(4-pyridinyl)isoxazol-5-yl]vinyl]-3-cephem-4-carboxylate
16) para-Methoxybenzyl(6R,7R)-7-phenylthioacetamido-3-[(3-carbamoylisoxazol-5-yl)vinyl]-3-cephem-4-carboxylate

### EXAMPLE 3

### Preparation of (6R,7R)-7-phenylthioacetamido-3-[(3-methylisoxazol-5-yl)vinyl]-3-cephem-4-carboxylic acid

para-Methoxybenzyl (6R,7R)-7-phenylthioacetamido-3-[(3-methylisoxazol-5-yl)vinyl]-3-cephem-4-carboxylate (164 mg, 0.28 mmol) was suspended in anisole (2.1 mL), and cooled to 0° C. To the suspension trifluoroacetic acid (2.1 mL) was added dropwise, and stirring was continued for 2 h at the same temperature. The reaction mixture was then concentrated under vacuum. The residue was solidified with petroleum ether, triturated, and filtered to give the title compound (81 mh, 62.3%).

¹H NMR (CD₃OD, 300 MHz, δ): 7.43 (d, 2H, J=7.2 Hz), 7.29 (m, 3H), 6.75 (d, 1H), 6.49 (d, 1H), 6.23 (s, 1H), 5.77 (d, 1H), 5.20 (d, 1H), 3.73 (d, 2H), 3.52 (dd, 2H), 2.27 (s, 3H)

### Preparation of (6R,7R)-7-(3,4-Dichlorophenyl)thioacetamido-3-[(3-methylisoxazol-5-yl)vinyl]-3-cephem-4-carboxylic acid

The title compound (120 mg, 80%) was obtained by following the same procedure as the above-mentioned Example 3 using the corresponding amount of para-methoxybenzyl(6R,7R)-7-(3,4-dichlorophenyl)thioacetamido-3-[(3-methylisoxazol-5-yl)vinyl]-3-cephem-4-carboxylate as the reactant.

¹H NMR (CD₃OD, 300 MHz, δ): 7.43 (s, 1H), 7.29 (m, 2H), 6.75 (d, 1H), 6.49 (d, 1H), 6.23 (s, 1H), 5.77 (d, 1H), 5.20 (d, 1H), 3.73 (d, 2H), 3.52 (dd, 2H), 2.27(s, 3H)

### Preparation of (6R,7R)-7-(3,5-Dimethylphenyl)thioacetamido-3-[(3-methylisoxazol-5-yl)vinyl]-3-cephem-4-carboxylic acid

The title compound (110 mg, 85%) was obtained by following the same procedure as the above-mentioned Example 3 using the corresponding amount of para-methoxybenzyl(6R,7R)-7-(3,5-dimethylphenyl)thioacetamido-3-[(3-methylisoxazol-5-yl)vinyl]-3-cephem-4-carboxylate as the reactant.

¹H NMR (CD₃OD, 300 MHz, δ): 7.35 (m, 3H), 6.75 (d, 1H), 6.49 (d, 1H), 6.23 (s, 1H), 5.77 (d, 1H), 5.20 (d, 1H), 3.73 (d, 2H), 3.52 (dd, 2H), 2.27(s, 3H)

### EXAMPLE 4

The following acid compounds were prepared by the same procedure as the Example 3 using the corresponding para-methoxybenzyl esters.
1) (6R,7R)-7-Phenylthioacetamido-3-[(3-ethylisoxazol-5-yl)vinyl]-3-cephem-4-carboxylic acid

¹H NMR (CD₃OD, 300 MHz, δ): 7.43 (d, 2H), 7.26 (m, 3H), 6.75 (d, 1H), 6.49 (d, 1H), 6.27 (s, 1H), 5.76 (d, 1H), 5.20 (d, 1H), 3.72 (d, 2H), 3.53 (dd, 2H), 2.68 (q, 2H), 1.26 (t, 3H)
2) (6R,7R)-7-Phenylthioacetamido-3-[(3-methoxyisoxazol-5-yl)vinyl]-3-cephem-4-carboxylic acid

¹H NMR (CD₃OD, 300 MHz, δ): 7.43 (d, 2H), 7.30 (m, 3H), 6.75 (d, 1H), 6.39 (d, 1H), 5.98 (s, 1H), 5.76 (d, 1H), 5.18 (d, 1H), 3.94 (s, 3H), 3.72 (s, 2H), 3.52 (dd, 2H)
3) (6R,7R)-7-Phenylthioacetamido-3-[(3-ethoxyisoxazol-5-yl)vinyl]-3-cephem-4-carboxylic acid

¹H NMR (CD₃OD, 300 MHz, δ): 7.43 (d, 2H), 7.31 (t, 2H), 7.23 (d, 1H), 6.75 (d, 1H), 6.40 (d, 1H), 5.97 (s, 1H), 5.77 (d, 1H), 5.19 (d, 1H), 4.26 (q, 2H), 3.72 (d, 2H), 3.53 (dd, 2H), 1.39 (t, 3H)
4) (6R,7R)-7-Phenylthioacetamido-3-[(3-bromoisoxazol-5-yl)vinyl]-3-cephem-4-carboxylic acid

¹H NMR (CD₃OD, 300 MHz, δ): 7.44 (d, 2H), 7.31 (t, 2H), 7.22 (t, 1H), 6.84 (d, 1H), 6.52 (d, 1H), 6.49 (s, 1H), 5.78 (d, 1H), 5.20 (d, 1H), 3.72 (d, 2H), 3.52 (dd, 2H)
5) (6R,7R)-7-Phenylthioacetamido-3-[(3-hydroxyisoxazol-5-yl)vinyl]-3-cephem-4-carboxylic acid

¹H NMR (DMSO-d₆, 300 MHz, δ): 11.30 (s, 1H), 9.22 (d, 1H), 7.29 (m, 5H), 6.64 (d, 1H), 6.37 (d, 1H), 5.92 (s, 1H), 5.73 (dd, 1H), 5.19 (d, 1H), 3.76 (d, 2H), 3.59 (m, 2H)
6) (6R,7R)-7-Phenylthioacetamido-3-[(3-ethoxycarbonylisoxazol-5-yl)vinyl]-3-cephem-4-carboxylic acid

¹H NMR (CD₃OD, 300 MHz, δ): 11.30 (s, 1H), 9.22 (d, 1H), 7.29 (m, 5H), 6.64 (d, 1H), 6.37 (d, 1H), 5.92 (s, 1H), 5.73 (dd, 1H), 5.19 (d, 1H), 3.76 (d, 2H), 3.59 (m, 2H)
7) (6R,7R)-7-Phenylthioacetamido-3-[(3-phenylisoxazol-5-yl)vinyl]-3-cephem-4-carboxylic acid

¹H NMR (CDCl3, 300 MHz, δ): 7.47 (m, 10H), 6.81 (d, 1H), 6.51 (d, 1H), 6.51 (s, 1H), 6.88 (dd, 1H), 5.12 (d, 1H), 3.73 (q, 2H), 3.51 (dd, 1H)
8) (6R,7R)-7-Phenylthioacetamido-3-[[3-(4-methylphenyl)isoxazol-5-yl]vinyl]-3-cephem-4-carboxylic acid

¹H NMR (CD3OD, 300 MHz, δ): 7.72 (d, 2H), 7.42 (d, 2H), 7.25 (m, 5H), 7.81 (d, 1H), 6.74 (s, 1H), 6.58 (d, 1H), 5.76 (d, 1H), 5.23 (d, 1H), 3.73 (d, 2H), 3.57 (dd, 2H), 2.39 (s, 3H)
9) (6R,7R)-7-Phenylthioacetamido-3-[[3-(ethoxyphenyl)isoxazol-5-yl]vinyl]-3-cephem-4-carboxylic acid

¹H NMR (CD3OD, 300 MHz, δ): 7.76 (d, 2H), 4.42 (d, 2H), 7.28 (t, 2H), 7.19 (t, 1H), 7.03 (d, 2H), 6.81 (d, 1H), 6.71 (s, 1H), 6.57 (d, 1H), 5.77 (d, 1H), 5.23 (d, 1H), 3.85 (s, 3H), 3.74 (d, 2H), 3.56 (dd, 2H)
10) (6R,7R)-7-Phenylthioacetamido-3-[[3-(4-fluorophenyl)isoxazol-5-yl]vinyl]-3-cephem-4-carboxylic acid

¹H NMR (CD3OD, 300 MHz, δ): 7.86 (dd, 2H), 7.41 (d, 2H), 7.23 (m, 5H), 6.81 (d, 1H), 6.75 (s, 1H), 6.57 (d, 1H), 5.75 (d, 1H), 5.22 (d, 1H), 3.72 (d, 2H), 3.56 (dd, 2H)
11) (6R,7R)-7-Phenylthioacetamido-3-[[3-(4-chlorophenyl)isoxazol-5-yl]vinyl]-3-cephem-4-carboxylic acid

¹H NMR (CD3OD, 300 MHz, δ): 7.84 (d, 2H), 7.51 (d, 2H), 7.43 (d, 2H), 7.29 (t, 2H), 7.19 (t, 1H), 6.83 (d, 1H), 6.78 (s, 1H), 6.59 (d, 1H), 5.77 (d, 1H), 5.23 (d, 1H), 3.73 (d, 2H), 3.57 (dd, 2H)
12) (6R,7R)-7-Phenylthioacetamido-3-[[3-(4-bromophenyl)isoxazol-5-yl]vinyl]-3-cephem-4-carboxylic acid

¹H NMR (CD3OD, 300 MHz, δ): 7.81 (d, 2H), 7.50 (d, 2H), 7.39 (d, 2H), 7.30 (t, 2H), 7.15 (t, 1H), 6.83 (d, 1H), 6.75 (s, 1H), 6.57 (d, 1H), 5.76 (d, 1H), 5.21 (d, 1H), 3.75 (d, 2H), 3.56 (dd, 2H)
13) (6R,7R)-7-Phenylthioacetamido-3-[[3-(2-pyridinyl)isoxazol-5-yl]vinyl]-3-cephem-4-carboxylic acid

¹H NMR (CD3OD, 300 MHz, δ): 8.69 (d, 1H), 8.15 (d, 1H), 8.05 (t, 1H), 7.58 (t, 1H), 7.41 (d, 2H), 7.27 (t, 2H), 7.19 (d, 1H), 6.97 (s, 1H), 6.86 (d, 1H), 6.63 (d, 1H), 5.79 (d, 1H), 5.25 (d, 1H), 3.74 (d, 2H), 3.57 (dd, 2H)
14) (6R,7R)-7-Phenylthioacetamido-3-[[3-(3-pyridinyl)isoxazol-5-yl]vinyl]-3-cephem-4-carboxylic acid

¹H NMR (CD3OD, 300 MHz, δ): 9.15 (s, 1H), 8.78 (d, 1H), 7.85 (d, 1H), 7.45 (t, 1H), 7.39 (d, 2H), 7.28 (t, 2H), 7.16 (d, 1), 6.87 (d, 1H), 6.68 (d, 1H), 6.45 (s, 1H), 5.79 (d, 1H), 5.25 (d, 1H), 3.75 (d, 2H), 3.56 (dd, 2H)
15) (6R,7R)-7-Phenylthioacetamido-3-[[3-(4-pyridinyl)isoxazol-5-yl]vinyl]-3-cephem-4-carboxylic acid

¹H NMR (CD3OD, 300 MHz, δ): 8.82 (dd, 2H), 8.34 (d, 1H), 8.22 (d, 1H), 7.43 (d, 2H), 7.27 (t, 2H), 7.21 (d, 1H), 7.02 (s, 1H), 6.91 (d, 1H), 6.63 (d, 1H), 5.74 (d, 1H), 5.38 (d, 1H), 3.74 (d, 2H), 3.61 (dd, 2H)
16) (6R,7R)-7-Phenylthioacetamido-3-[(3-carbamoylisoxazol-5-yl)vinyl]-3-cephem-4-carboxylic acid

¹H NMR (CD₃OD, 300 MHz, δ): 7.43 (d, 2H), 7.29 (m, 3H), 6.84 (d, 1H), 6.67 (s, 1H), 6.58 (d, 1H), 5.79 (d, 1H), 5.21 (d, 1H), 3.73 (d, 2H), 3.52 (dd, 2H)

### EXAMPLE 5

An example for introducing ester residue at R₂ to prepare carboxylic ester derivatives that show in vivo antibacterial activity

### Preparation of (R,S)-1-(isopropoxycarbonyloxy)ethyl(6R,7R)-7-phenylthioacetamido-3-[(3-methylisoxazol-5-yl)vinyl]-3-cephem-4-carboxylate

(6R,7R)-7-Phenylthioacetamido-3-[(3-methylisoxazol-5-yl)vinyl]-3-cephem-4-carboxylic acid (0.5 g, 0.93 mmol) was dissolved in N,N-dimethylacetamide (30 mL), and cooled to −15° C.˜−10° C. To the solution was added 1,8-diazabicyclo[5.4.0]undec-7-ene (0.14 g, 0.93 mmol) and 1-iodoethyl isopropyl carbonate (10 mL), and the reaction mixture was stirred for 3 h. After completion of the reaction, the precipitate produced by the addition of ethyl acetate (100 mL) was filtered off. To the filtrate was successively added dilute hydrochloric acid and 5% aqueous sodium bicarbonate. The organic layer was separated, washed with water and brine, dried with anhydrous magnesium sulfate, and filtered. The filtrate was concentrated under vacuum, and the residue was column chromatographed on silica gel, eluting with a 3:2 mixture of ethyl acetate and n-hexane, to provide the title compound (450 mg, 85.7%).

¹H NMR (CD₃OD, 300 MHz, δ): 7.43 (d, 2H, J=7.2 Hz), 7.29 (m, 3H), 6.75 (d, 1H), 6.61 (m, 1H), 6.49 (d, 1H), 6.23 (s, 1H), 5.77 (d, 1H), 5.20 (d, 1H), 4.31 (m, 1H), 3.73 (d, 2H), 3.52(dd, 2H), 2.27(s,3H), 1.74(d, 3H), 1.35(d, 6H)

### TEST EXAMPLE 1

### In Vitro Antibacterial Activity Test

The in vitro antibacterial activity of the compounds prepared in the Examples of the invention was determined as follows: after incubation of the corresponding bacterium for 18 h at 37° C., minimum inhibitory concentrations (MICs, μg/mL) were determined by the 2-fold agar dilution method with Mueller-Hinton Agar. Some of the are summarized in the following Table 1.

TABLE 1
Minimum inhibitory concentrations of representative compounds (MICs, μg/mL)

| | | Compound | Compound | Compound | | |
|---|---|---|---|---|---|---|
| | MRSA | 24 | 28 | 30 | cefotaxime | vancomycin |
| 1 | Staphylococcus | 1.563 | 3.125 | 3.125 | 25.000 | 3.125 |
| | aureus 001 | | | | | |
| 2 | Staphylococcus | 0.007 | <0.002 | 0.002 | 3.125 | 1.563 |
| | aureus 002 | | | | | |
| 3 | Staphylococcus | 3.125 | 12.500 | 6.250 | >100.000 | 0.781 |
| | aureus 003 | | | | | |
| 4 | Staphylococcus | 6.250 | 6.250 | 3.125 | >10.000 | 0.781 |
| | aureus 004 | | | | | |
| 5 | Staphylococcus | 0.098 | 0.098 | 0.098 | 6.250 | 0.781 |
| | aureus 005 | | | | | |
| 6 | Staphylococcus | 0.013 | 0.013 | 0.004 | 1.563 | 0.781 |
| | aureus 006 | | | | | |
| 7 | Staphylococcus | 0.013 | 0.013 | <0.002 | 1.563 | 0.781 |
| | aureus 007 | | | | | |
| 8 | Staphylococcus | 0.098 | 0.195 | 0.195 | 1.563 | 0.781 |
| | aureus 008 | | | | | |
| 9 | Staphylococcus | 0.049 | 0.195 | 0.195 | 6.250 | 0.781 |
| | aureus 009 | | | | | |
| 10 | Staphylococcus | 0.391 | 0.391 | 0.195 | 6.250 | 0.781 |
| | aureus 010 | | | | | |

As illustrated in Table 1, the compounds of the invention exhibit excellent antibacterial activity against methicillin-resistant Staphylococcus aureus.

### INDUSTRIAL APPLICABILITY

As apparent from the foregoing, since the cephalosporin compounds of the present invention show superior antibacterial activity against a wide variety of gram-positive bacteria, particularly methicillin-resistant Staphylococcus aureus train, they can be effectively used as antibiotics.

In addition, according to the method of the present invention, high purity cephalosporin compounds can be prepared in high yield.

1. A cephalosporin compound, or a pharmaceutically acceptable salt thereof, represented by Formula I below: 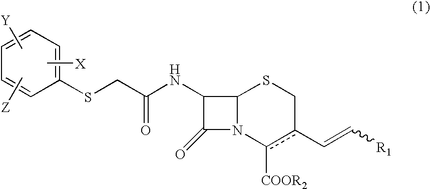
wherein X, Y and Z may be the same or different from one another, and are each independently hydrogen, halogen, C₁₋₆ alkyl, Clue alkoxy, C₁₋₆ halogenoalkyl, C₁₋₆ alkoxyalkyl, or C₃₋₆ cycloalkyl;
R₁ is a 3-substituted isoxazolyl group represented by Formula A below: 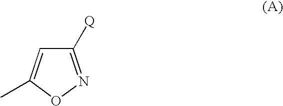
(wherein Q is a substituent useful for the cephalosporin compounds, and is hydrogen, halogen, hydroxy, mercapto, cyano, carboxy, carboxylic acid, ester, carbamoyloxy, carbamoyl, N,N-dimethylcarbamoyl, C₁₋₄ alkyl, C₁₋₄ alkyloxy, halogen-substituted alkyl, aryl, or heterocyclic group); and
R₂ is hydrogen, a group forming an ester as a carboxyl derivative, a salt-forming element, or a carboxy-protecting group.

2. The compound according to claim 1, wherein the compound is selected from the group consisting of:
para-methoxybenzyl(6R,7R)-7-phenylthioacetamido-3-[(3-methylisoxazol-5-yl)vinyl]-3-cephem-4-carboxylate;
para-methoxybenzyl(6R,7R)-7-phenylthioacetamido-3-[(3-ethylisoxazol-5-yl)vinyl]-3-cephem-4-carboxylate;
para-methoxybenzyl(6R,7R)-7-phenylthioacetamido-3-[(3-methoxyisoxazol-5-yl)vinyl]-3-cephem-4-carboxylate;
para-methoxybenzyl(6R,7R)-7-phenylthioacetamido-3-[(3-ethoxyisoxazol-5-yl)vinyl]-3-cephem-4-carboxylate;
para-methoxybenzyl(6R,7R)-7-phenylthioacetamido-3-[(3-bromoisoxazol-5-yl)vinyl]-3-cephem-4-carboxylate;
para-methoxybenzyl(6R,7R)-7-phenylthioacetamido-3-[(3-hydroxyisoxazol-5-yl)vinyl]-3-cephem-4-carboxylate;
para-methoxybenzyl(6R,7R)-7-phenylthioacetamido-3-[(3-ethoxycarbonylisoxazol-5-yl)vinyl]-3-cephem-4-carboxylate;
para-methoxybenzyl(6R,7R)-7-phenylthioacetamido-3-[(3-phenylisoxazol-5-yl)vinyl]-3-cephem-4-carboxylate;
para-methoxybenzyl(6R,7R)-7-phenylthioacetamido-3-[[3-(4-methylphenyl)isoxazol-5-yl]vinyl]-3-cephem-4-carboxylate;
para-methoxybenzyl(6R,7R)-7-phenylthioacetamido-3-[[3-(4-methoxyphenyl)isoxazol-5-yl]vinyl]-3-cephem-4-carboxylate;
para-methoxybenzyl(6R,7R)-7-phenylthioacetamido-3-[[3-(4-fluorophenyl)isoxazol-5-yl]vinyl]-3-cephem-4-carboxylate;
para-methoxybenzyl(6R,7R)-7-phenylthioacetamido-3-[[3-(4-chlorophenyl)isoxazol-5-yl]vinyl]-3-cephem-4-carboxylate;
para-methoxybenzyl(6R,7R)-7-phenylthioacetamido-3-[[3-(4-bromophenyl)isoxazol-5-yl)vinyl]-3-cephem-4-carboxylate;
para-methoxybenzyl(6R,7R)-7-phenylthioacetamido-3-[[3-(pyridin-2-yl)isoxazol-5-yl]vinyl]-3-cephem-4-carboxylate;
para-methoxybenzyl (6R,7R)-7-phenylthioacetamido-3-([3-(pyridin-3-yl)isoxazol-5-yl]vinyl]-3-cephem-4-carboxylate;
para-methoxybenzyl (6R,7R)-7-phenylthioacetamido-3-[[3-(pyridin-4-yl)isoxazol-5-yl]vinyl]-3-cephem-4-carboxylate;
para-methoxybenzyl (6R,7R)-7-phenylthioacetamido-3-[(3 carbamoylisoxazol-5-yl)vinyl]-3-cephem-4-carboxylate;
(6R,7R)-7-phenylthioacetamido-3-[(3-methylisoxazol-5-yl)vinyl]-3-cephem-4-carboxylic acid;
(6R,7R)-7-phenylthioacetamido-3-[(3-ethylisoxazol-5-yl)vinyl]-3-cephem-4-carboxylic acid;
(6R,7R)-7-phenylthioacetamido-3-[(3-methoxyisoxazol-5-yl)vinyl]-3-cephem-4-carboxylic acid;
(6R,7R)-7-phenylthioacetamido-3-[(3-ethoxyisoxazol-5-yl)vinyl]-3-cephem-4-carboxylic acid;
(6R,7R)-7-phenylthioacetamido-3-[(3-bromoisoxazol-5-yl)vinyl]-3-cephem-4-carboxylic acid;
(6R,7R)-7-phenylthioacetamido-3-[(3-hydroxyisoxazol-5-yl)vinyl]-3-cephem-4-carboxylic acid;
(6R,7R)-7-phenylthioacetamido-3-[(3-ethoxycarbonylisoxazol-5-yl)vinyl]-3-cephem-4-carboxylic acid;
(6R,7R)-7-phenylthioacetamido-3-[(3-phenylisoxazol-5-yl)vinyl]-3-cephem-4-carboxylic acid;
(6R,7R)-7-phenylthioacetamido-3-[[3-(4-methylphenyl)isoxazol-5-yl]vinyl]-3-cephem-4-carboxylic acid;
(6R,7R)-7-phenylthioacetamido-3-[[3-(4-methoxyphenyl)isoxazol-5-yl]vinyl]-3-cephem-4-carboxylic acid;
(6R,7R)-7-phenylthioacetamido-3-[[3-4-fluorophenyl)isoxazol-5-yl]vinyl]-3-cephem-4-carboxylic acid;
(6R,7R)-7-phenylthioacetamido-3-[[3-(4-chlorophenyl)isoxazol-5-yl]vinyl]-3-cephem-4-carboxylic acid;
(6R,7R)-7-phenylthioacetamido-3-[[3-(4-bromophenyl)isoxazol-5-yl]vinyl]-3-cephem-4-carboxylic acid;
(6R,7R)-7-phenylthioacetamido-3-[[3-(pyridin-2-yl)isoxazol-5-yl]vinyl]-3-cephem-4-carboxylic acid;
(6R,7R)-7-phenylthioacetamido-3-[[3-(pyridin-3-yl)isoxazol-5-yl]vinyl]-3-cephem-4-carboxylic acid;
(6R,7R)-7-phenylthioacetamido-3-[[3-(pyridin-4-yl)isoxazol-5-yl]vinyl]-3-cephem-4-carboxylic acid; and
(6R,7R)-7-phenylthioacetamido-3-[(3-carbamoylisoxazol-5-yl)vinyl]-3-cephem-4-carboxylic acid.

3. A method for preparing a cephalosporin compound or a pharmaceutically acceptable salt thereof, comprising the steps:
preparing a compound represented by Formula I below: 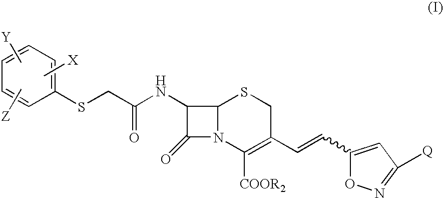
wherein X, Y and Z may be the same or different from one another, and are each independently hydrogen, halogen, C₁₋₆ alkyl, C₁₋₆ alkoxy, C₁₋₆ halogenoalkyl, C₁₋₆ alkoxyalkyl, or C₃₋₆ cycloalkyl;
Q is a substituent useful for the cephalosporin compound, and is hydrogen, halogen, hydroxy, mercapto, cyano, carboxy, carboxylic acid, ester, carbamoyloxy, carbamoyl, N,N-dimethylcarbamoyl, C₁₋₄ alkyl, C₁₋₄ alkyloxy, halogen-substituted alkyl, aryl, or heterocyclic group; and
R₂ is hydrogen, a group forming an ester as a carboxyl derivative, a salt-forming element, or a carboxy-protecting group,
by reacting an ylide of Formula VI below: 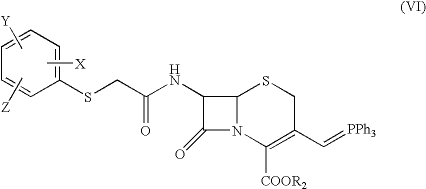
wherein X Y, Z and R₂ are as defined above,
with an aldehyde compound of Formula VII below: 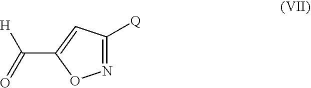
wherein Q is as defined above,
in the presence of a base and an organic solvent.

4. The method according to claim 3, wherein the base is at least one selected from the group consisting of sodium carbonate, sodium hydrogen carbonate, alkali metal hydride, alkali metal amide, alkali metal hydroxide, alkali metal acetate, tri-(lower)alkylbenzylamine, N-lower alkylmorpholine, N,N-(lower)alkylbenzylamine and N,N-di-(lower)alkylaniline.

5. The method according to claim 3 or 4, wherein the solvent is at least one selected from the group consisting of water, acetone, dioxane, acetonitrile, chloroform, dichloromethane, tetrahydrofuran, ethylacetate and N,N-dimethylformamide.

6. The method according to claim 3 or 4, wherein the reaction is carried out at a temperature between −40° C. and 25° C.

7. The method according to claim 3 or 4, further comprising removing the protecting group by reacting the compound of Formula I with an acid, as depicted in Reaction Scheme 4 below: 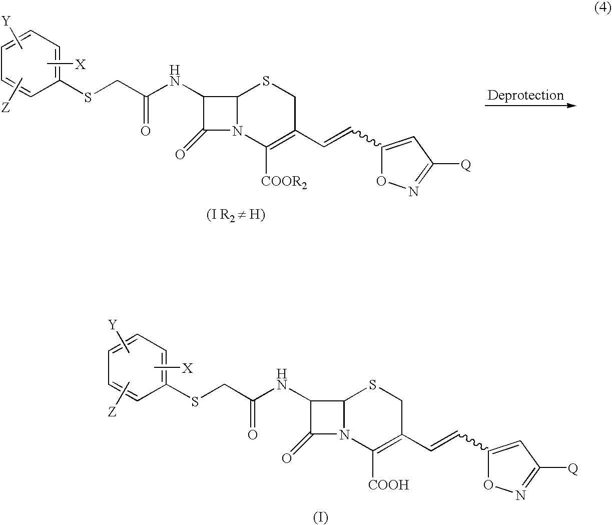
wherein X, Y and Z may be the same or different from one another, and are each independently hydrogen, halogen, C₁₋₆ alkyl, C₁₋₆ alkoxy, C₁₋₆ halogenoalkyl, C₁₋₆ alkoxyalkyl, or C₃₋₆ cycloalkyl;
Q is a substituent useful for the cephalosporin compound, and is hydrogen, halogen, hydroxy, mercapto, cyano, carboxy, carboxylic acid, ester, carbamoyloxy, carbamoyl, N,N-dimethylcarbamoyl, C₁₋₄ alkyl, C₁₋₄ alkyloxy, halogen-substituted alkyl, aryl, or heterocyclic group; and
R₂ is hydrogen, a group forming an ester as a carboxyl derivative, a salt-forming element, or a carboxy-protecting group.

8. An antibiotic composition, comprising:
the cephalosporin compound or its pharmaceutically acceptable salt according to claim 1; and
a pharmaceutically acceptable carrier.
